(19) United States
(12) Patent Application Publication
(10) Pub. No.: US 2021/0378781 A1
(43) Pub. Date: Dec. 9, 2021

(54) SYSTEMS AND METHODS FOR DISTINGUISHING KINEMATIC CHAINS IN ROBOTIC SURGERY

(71) Applicant: Auris Health, Inc., Redwood City, CA (US)

(72) Inventor: Fabien Y. Schmitt, San Jose, CA (US)

(21) Appl. No.: 17/339,908

(22) Filed: Jun. 4, 2021

Related U.S. Application Data
(60) Provisional application No. 63/035,305, filed on Jun. 5, 2020.

(51) Int. Cl. A61B 90/00; A61B 34/30; A61B 90/92

(52) U.S. Cl. CPC A61B 90/08; A61B 34/30; A61B 90/92; A61B 34/37; A61B 2034/302; A61B 2034/303; A61B 2034/301; A61B 2090/0807

(57) ABSTRACT

A surgical system can include a master controller for controlling one or more surgical tools. The system can also include an input on the master controller configured to change the master controller from a first mode into a second mode. The first mode can be a teleoperation mode and the second mode can be a virtual marking mode. In the virtual marking mode, a user is capable of communicating a virtual marker to other staff.

### CROSS-REFERENCE TO RELATED APPLICATIONS

This application claims priority to and the benefit of U.S. Provisional App. No. 63/035,305, filed Jun. 5, 2020, which is hereby incorporated by reference in its entirety.

### TECHNICAL FIELD

This application is directed to robotic medical systems, and more particularly to indicators configured for use with robotic medical systems.

### BACKGROUND

Medical procedures, such as laparoscopy or endoscopy, may involve accessing and visualizing an internal region of a patient. In a laparoscopic procedure, for example, a medical instrument can be inserted into an internal region through a laparoscopic access port. Robotically enabled medical system can be used to perform such medical procedures. The robotically enabled medical systems may include several robotic components, including, for example, robotic arms, robotic instrument manipulators, and robotic medical instruments, such as robotically controllable laparoscopes or endoscopes. The robotically enabled medical systems can be controlled using a user console that may include one or more hand operated inputs as well as one or more foot operated inputs.

### SUMMARY

The systems, methods, and devices of this disclosure each have several innovative aspects, no single one of which is solely responsible for the desirable attributes disclosed herein.

In a first aspect, a medical robotic system includes a first kinematic chain and a second kinematic chain. The first kinematic chain can include a first set of indicators and the second kinematic chain can include a second set of indicators. Actuation of the first set of indicators or the second set of indicators can differentiate the first kinematic chain from the second kinematic chain. The first kinematic chain and the second kinematic chain can have an equal number of degrees of freedom. The equal number of degrees of freedom can be at least 7 degrees of freedom. The first kinematic chain can be a first robotic arm and the second kinematic chain cam be a second robotic arm. The first robotic arm and the second robotic arm can be supported on an arm support. The first set of indicators can be positioned at a distal end of the first kinematic chain and the second set of indicators can be positioned at a distal end of the second kinematic chain. The first set of indicators and the second set of indicators can each include two or more bands of light. The two or more bands of lights of the first set of indicators can each be positioned about a circumference of the first kinematic chain. The two or more bands of lights of the second set of indicators are each positioned about a circumference of the second kinematic chain. Each band of light can be visible at 360 degrees about its respective first kinematic chain and/or the second kinematic chain. On the first kinematic chain, the two or more bands of lights can be positioned between a roll joint and a pitch joint. The first kinematic chain can include six links and six joints. The first set of indicators can be positioned on a fifth link of the six links. The first set of indicators can be positioned between a fifth joint and a sixth joint of the six joints.

In another aspect, a medical robotic system can include a first robotic arm having a first set of indicators and a second robotic arm having a second set of indicators. Actuation of the first set of indicators and the second set of indicators can differentiate the first robotic arm from the second robotic arm. The first robotic arm and the second robotic arm can have an equal number of degrees of freedom. The equal number of degrees of freedom can be at least 7 degrees of freedom. The first robotic arm and the second robotic arm can be supported on an arm support. The first set of indicators and the second set of indicators each can include two or more bands of light emitting diodes.

In yet another aspect, a medical robotic system can include a plurality of robotic arms. Each of the plurality of robotic arms can include one or more ring indicators. Actuation of the plurality of indicators can differentiate each of the plurality of robotic arms. The one or more indicators can include a number of ring indicators equal to half a number of the plurality of arms. The one or more ring indicators can be configured to display multiple colors. The one or more ring indicators of each of the plurality of robotic arms can be configured to display a different color. The plurality of robotic arms can include a first set of robotic arms positioned on a first side of a bed. The plurality of robotic arms can also include a second set of robotic arms positioned on a second side of the bed. The one or more ring indicators of each of the first set of robotic arms can be configured to display a first color. The one or more ring indicators of each of the second set of robotic arms can be configured to display a second color, the second color different from the first color. Each of the first set of robotic arms can be configured to display a different number of ring indicators to differentiate each of the first set of robotic arms. The first set of robotic arms can include a first robotic arm, a second robotic arm, and a third robotic arm. The first robotic arm can be configured to display one ring indicator, the second robotic arm can be configured to display two ring indicators, and the third robotic arm can be configured to display three ring indicators. The one or more ring indicators of each of the plurality of robotic arms can be configured to be programmed by a user.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopic procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopic procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21A illustrates an example of a robotic arm with indicators in a first position.

FIG. 21B illustrates an example of the robotic arm with indicators of FIG. 21A in a second position.

FIG. 22 illustrates another example of a robotic arm with indicators.

FIG. 23A illustrates an example of a robotic arm with a plurality of indicators.

FIG. 23B illustrates another example of a robotic arm with an indicator.

FIG. 23C illustrates another example of a robotic arm with an indicator.

FIG. 24A illustrates an example of a robotic system with a plurality of robotic arms with indicators.

FIG. 24B illustrates another example of a robotic system with a plurality of robotic arms with indicators.

FIG. 25A illustrates yet another example of a robotic system with a plurality of robotic arms with indicators.

FIG. 25B illustrates an example of a robotic system with a plurality of robotic arms with a plurality of indicators.

FIG. 25C illustrates another example of a robotic system with a plurality of robotic arms with a plurality of indicators.

FIG. 25D illustrates yet another example of a robotic system with a plurality of robotic arms with a plurality of indicators.

FIG. 26 illustrates an example of a display including an image overlay.

### DETAILED DESCRIPTION

### 1. Overview

Aspects of the present disclosure may be integrated into a robotically enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

A. Robotic System—Cart.

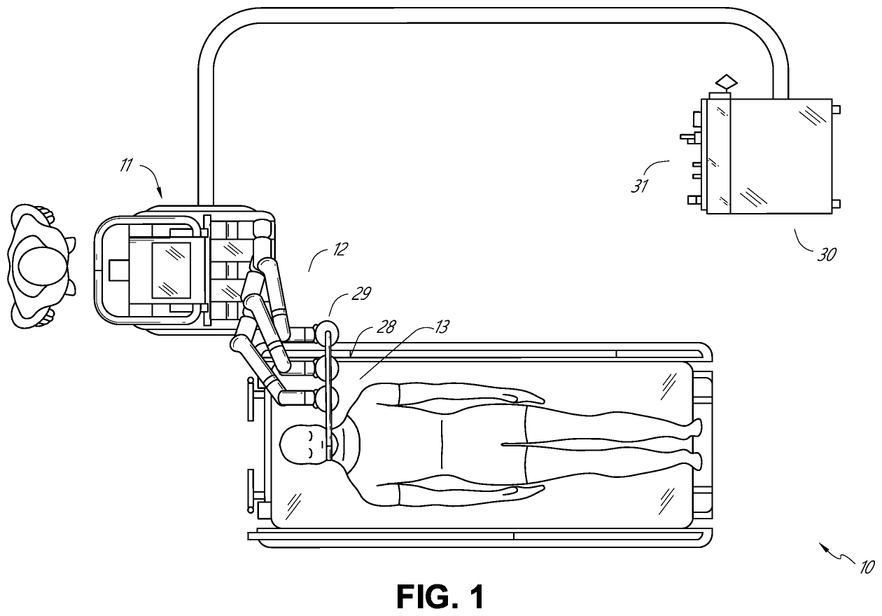

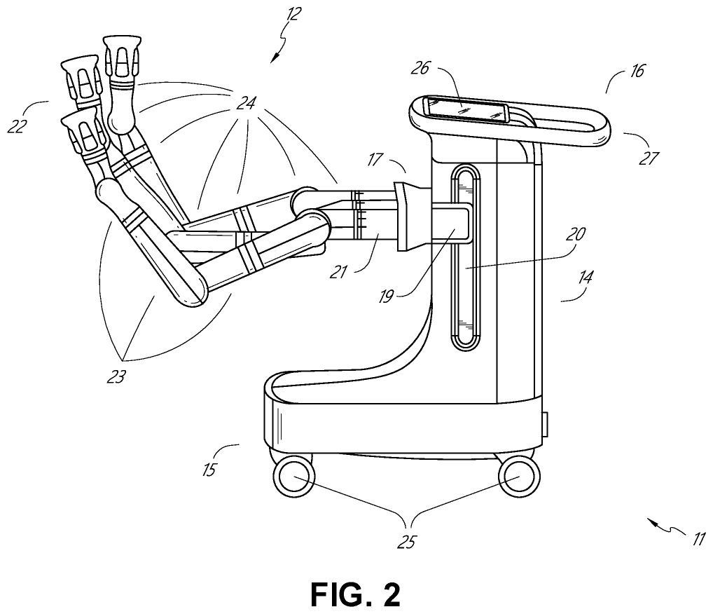

The robotically enabled medical system may be configured in a variety of ways depending on the particular procedure. FIG. 1 illustrates an embodiment of a cart-based robotically enabled system 10 arranged for a diagnostic and/or therapeutic bronchoscopy. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independently of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart 11, the support for controls, optics, fluidics, and/or navigation may be provided through a separate cable.

FIG. 2 provides a detailed illustration of an embodiment of the cart 11 from the cart-based robotically enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when the carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Having redundant degrees of freedom allows the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intraoperative data. Potential preoperative data on the touchscreen 26 may include preoperative plans, navigation and mapping data derived from preoperative computerized tomography (CT) scans, and/or notes from preoperative patient interviews. Intraoperative data on display may include optical information provided from the tool, sensor and coordinate information from sensors, as well as vital patient statistics, such as respiration, heart rate, and/or pulse. The console 16 may be positioned and tilted to allow a physician to access the console 16 from the side of the column 14 opposite the carriage 17. From this position, the physician may view the console 16, robotic arms 12, and patient while operating the console 16 from behind the cart 11. As shown, the console 16 also includes a handle 27 to assist with maneuvering and stabilizing the cart 11.

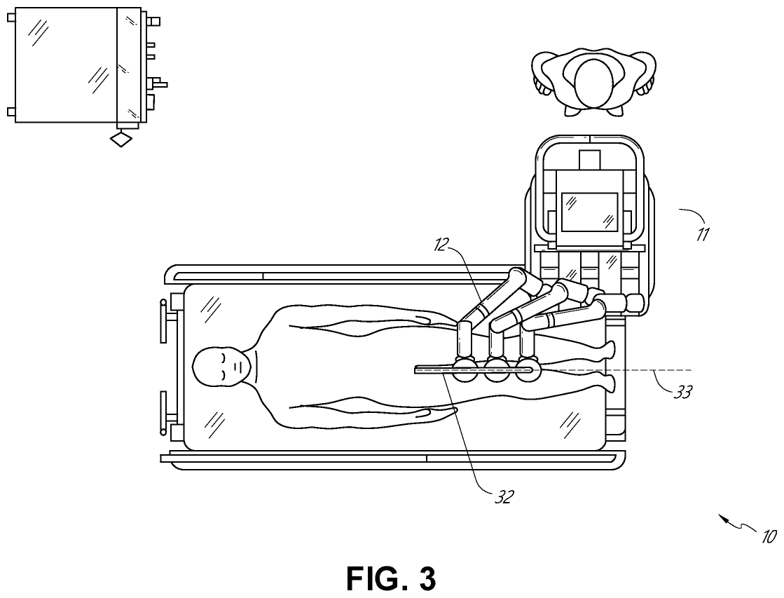

FIG. 3 illustrates an embodiment of a robotically enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

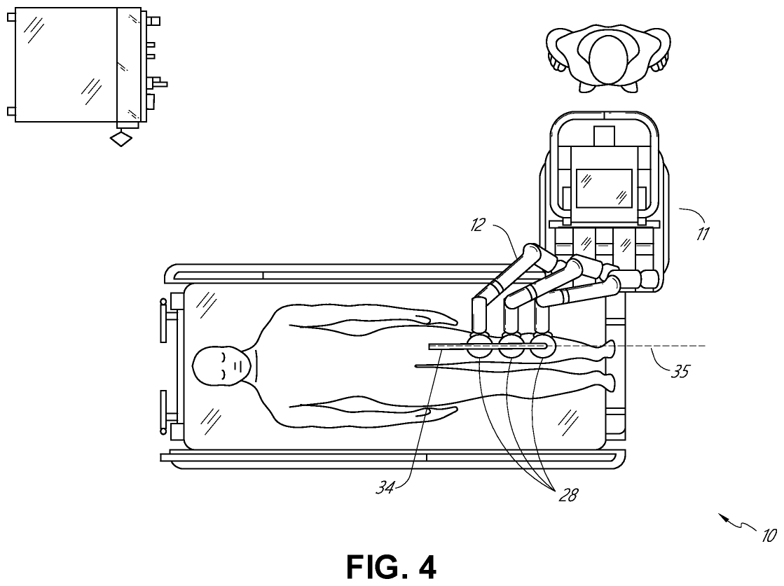

FIG. 4 illustrates an embodiment of a robotically enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

B. Robotic System—Table.

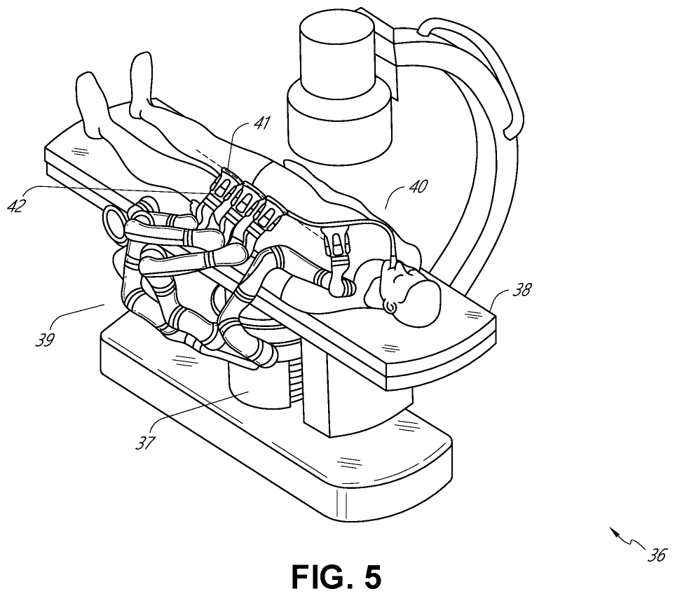

Embodiments of the robotically enabled medical system may also incorporate the patient's table. Incorporation of the table reduces the amount of capital equipment within the operating room by removing the cart, which allows greater access to the patient. FIG. 5 illustrates an embodiment of such a robotically enabled system arranged for a bronchoscopic procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

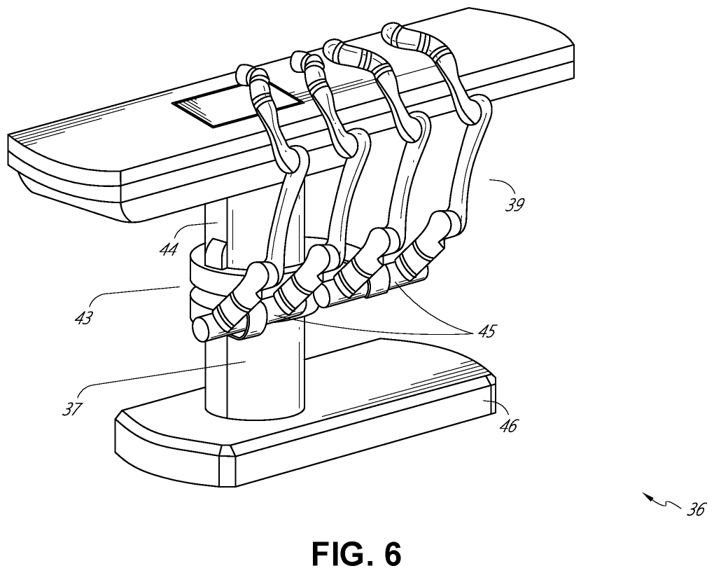

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independently of the other carriages. While the carriages 43 need not surround the column 37 or even be circular, the ring-shape as shown facilitates rotation of the carriages 43 around the column 37 while maintaining structural balance. Rotation and translation of the carriages 43 allows the system 36 to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

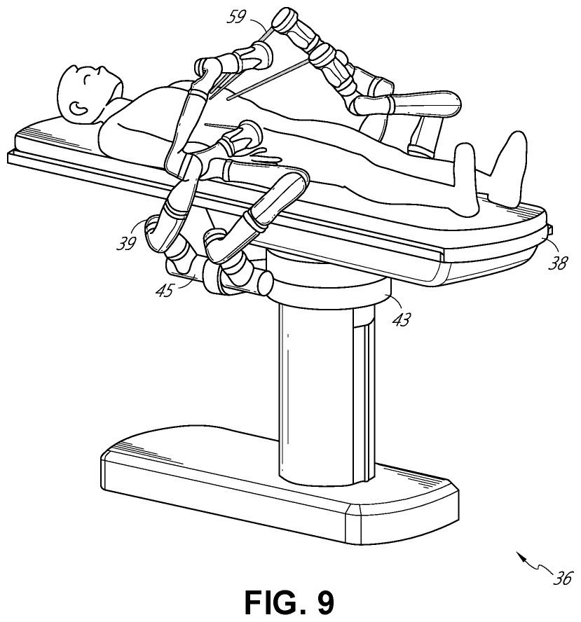

The robotic arms 39 may be mounted on the carriages 43 through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of the table 38 (as shown in FIG. 6), on opposite sides of the table 38 (as shown in FIG. 9), or on adjacent sides of the table 38 (not shown).

The column 37 structurally provides support for the table 38, and a path for vertical translation of the carriages 43. Internally, the column 37 may be equipped with lead screws for guiding vertical translation of the carriages, and motors to mechanize the translation of the carriages 43 based the lead screws. The column 37 may also convey power and control signals to the carriages 43 and the robotic arms 39 mounted thereon.

The table base 46 serves a similar function as the cart base 15 in the cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

With continued reference to FIG. 6, the system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 46 for potential stowage of the robotic arms 39. The tower may also include a master controller or console that provides both a user interface for user input, such as keyboard and/or pendant, as well as a display screen (or touchscreen) for preoperative and intraoperative information, such as real-time imaging, navigation, and tracking information. In some embodiments, the tower may also contain holders for gas tanks to be used for insufflation.

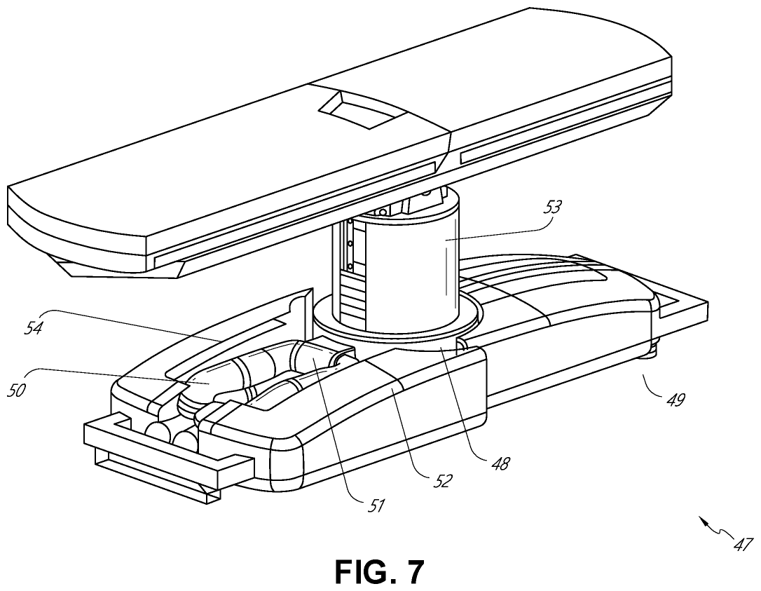

In some embodiments, a table base may stow and store the robotic arms when not in use. FIG. 7 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and robotic arms 50 around column 53, and closed to stow to protect them when not in use. The base covers 52 may be sealed with a membrane 54 along the edges of its opening to prevent dirt and fluid ingress when closed.

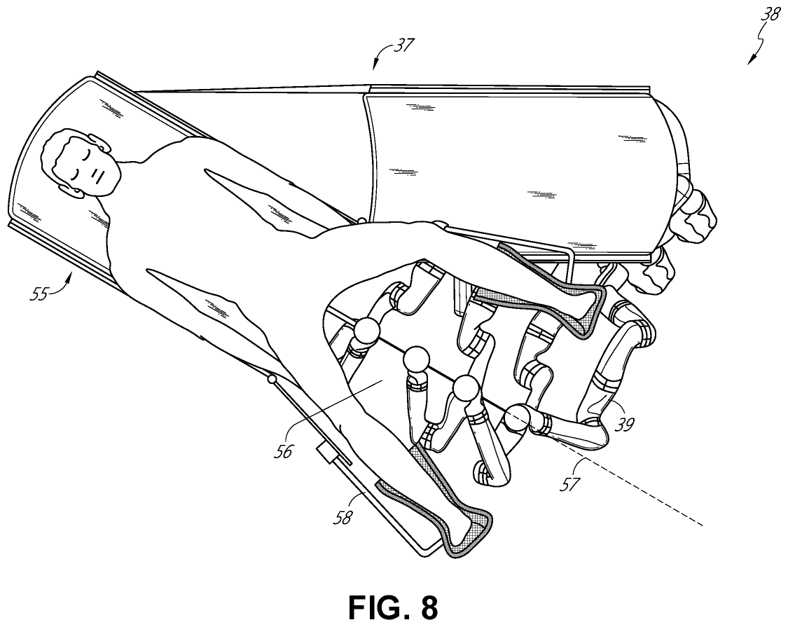

FIG. 8 illustrates an embodiment of a robotically enabled table-based system configured for a ureteroscopic procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

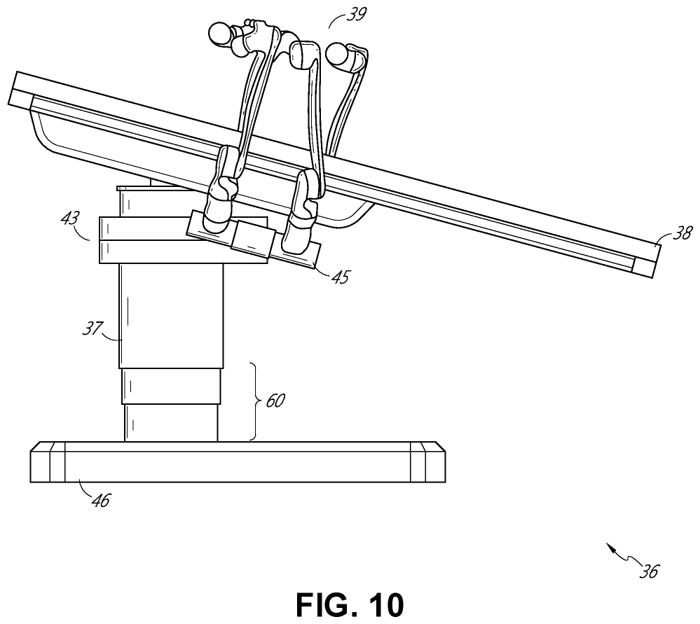

To accommodate laparoscopic procedures, the robotically enabled table system may also tilt the platform to a desired angle. FIG. 10 illustrates an embodiment of the robotically enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the robotic arms 39 maintain the same planar relationship with the table 38. To accommodate steeper angles, the column 37 may also include telescoping portions 60 that allow vertical extension of the column 37 to keep the table 38 from touching the floor or colliding with the table base 46.

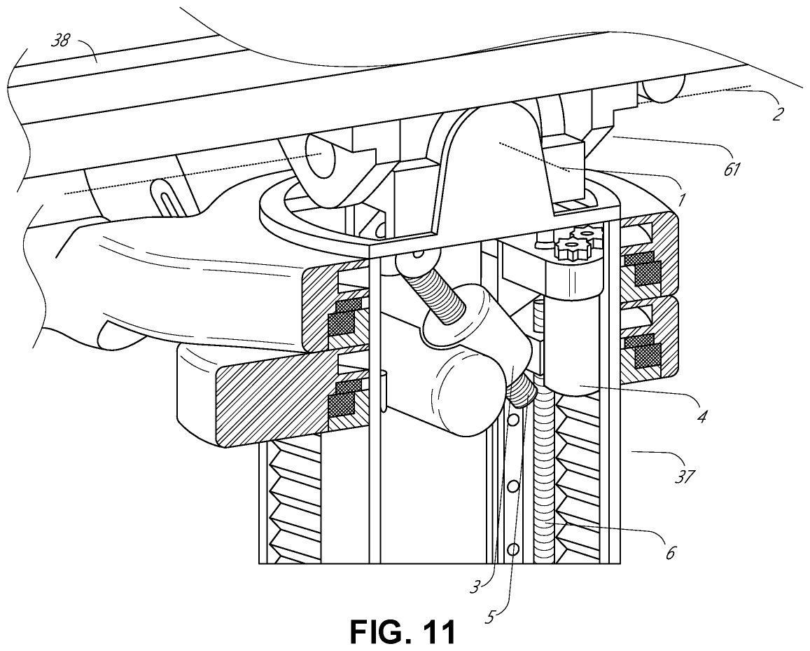

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's upper abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

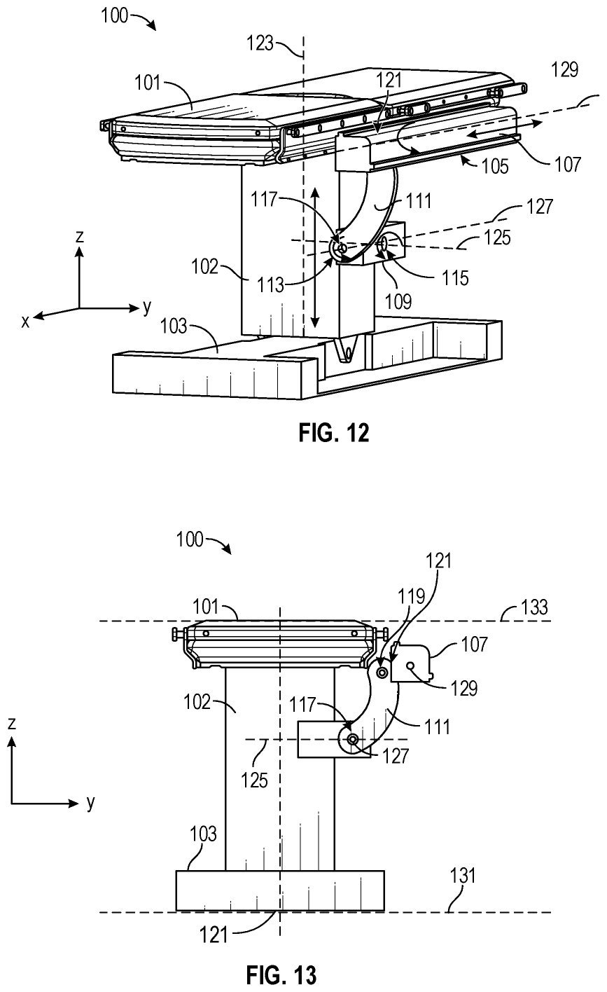

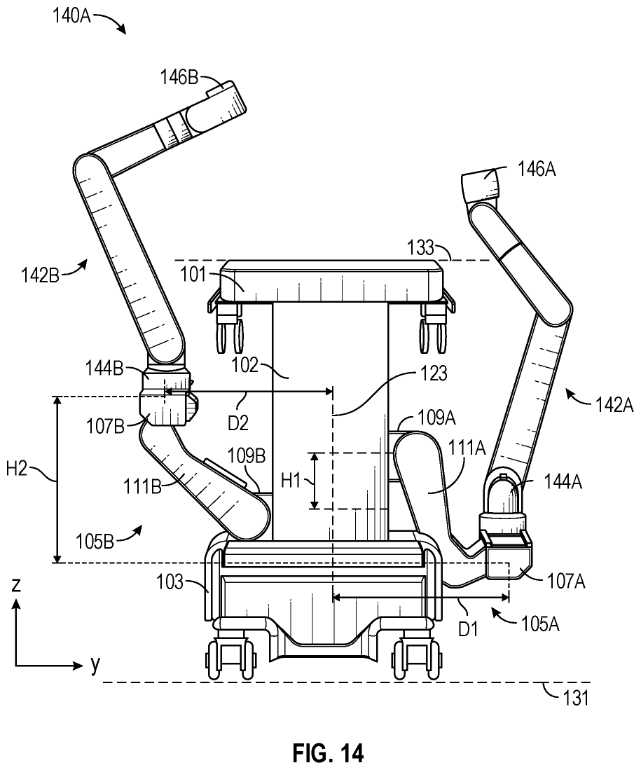

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (Z-lift) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

C. Instrument Driver & Interface.

The end effectors of the system's robotic arms may comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

FIG. 15 illustrates an example instrument driver. Positioned at the distal end of a robotic arm, instrument driver 62 comprises one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independently controlled and motorized, the instrument driver 62 may provide multiple (e.g., four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

D. Medical Instrument.

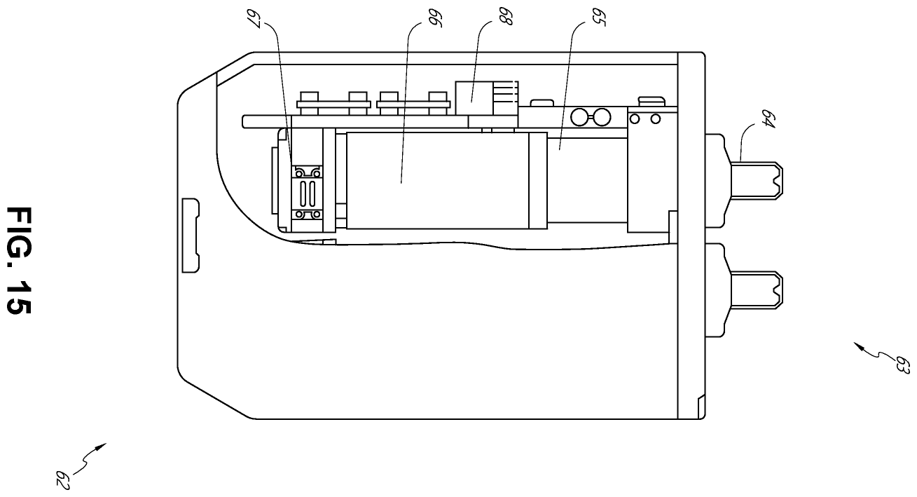

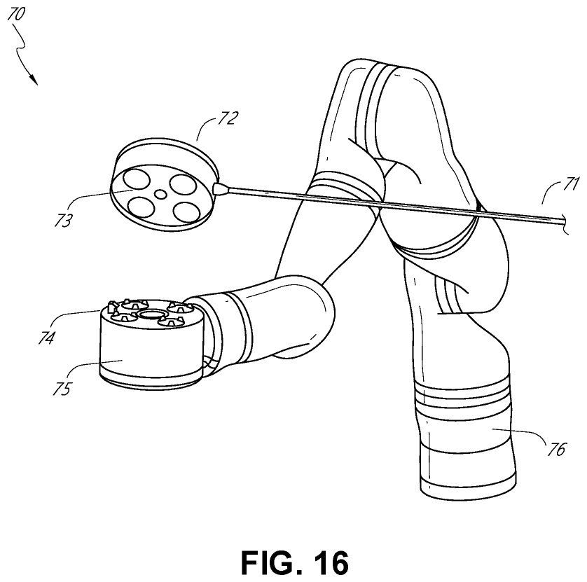

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of the instrument base 72 may share axes of rotation with the drive outputs 74 in the instrument driver 75 to allow the transfer of torque from the drive outputs 74 to the drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the elongated shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at the distal end of the elongated shaft 71, where tension from the tendon causes the grasper to close.

In endoscopy, the tendons may be coupled to a bending or articulating section positioned along the elongated shaft 71 (e.g., at the distal end) via an adhesive, a control ring, or other mechanical fixation. When fixedly attached to the distal end of a bending section, torque exerted on the drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft 71 may comprise a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include an optical camera. The shaft 71 may also accommodate optical fibers to carry light from proximally located light sources, such as light emitting diodes, to the distal end of the shaft 71.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft 71. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft 71 during an endoscopic procedure.

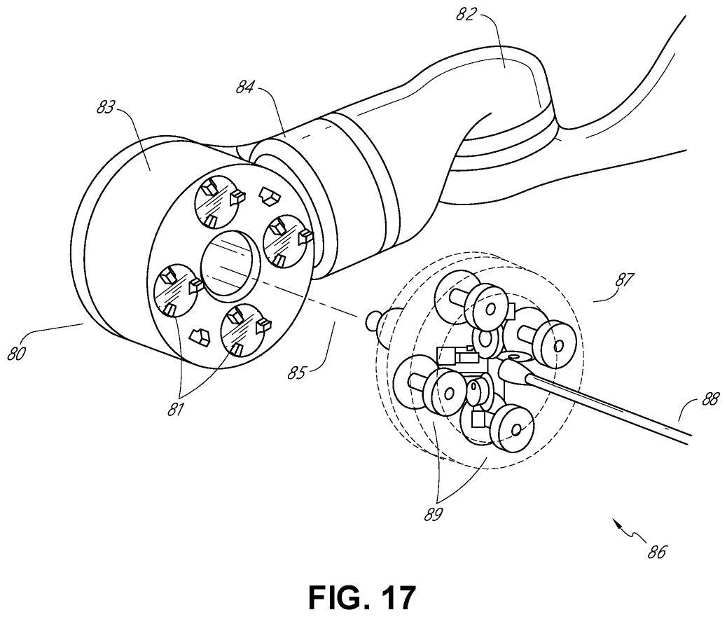

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver 80. Power and controls signals may be communicated from the non-rotational portion 84 of the instrument driver 80 to the rotational assembly 83 through electrical contacts that may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, the instrument shaft 88 extends from the center of the instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

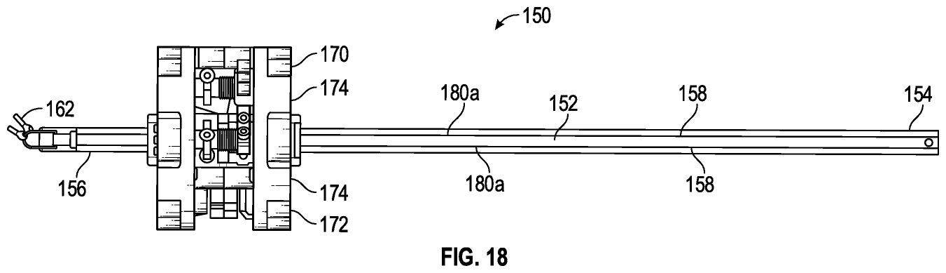

FIG. 18 illustrates an instrument having an instrument-based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

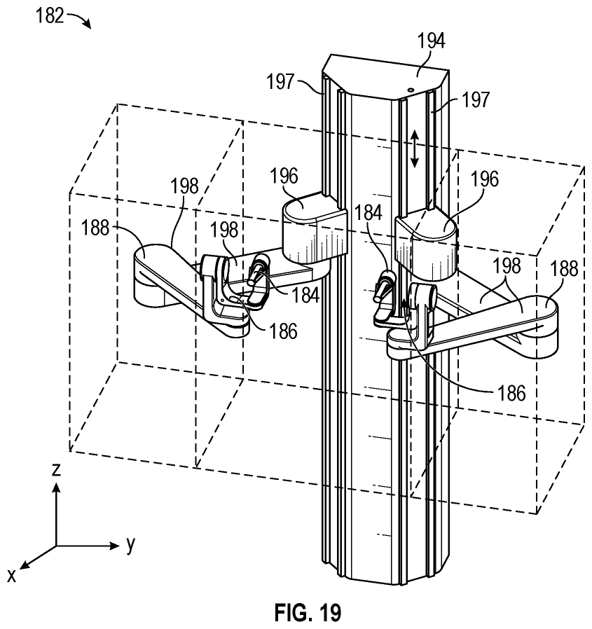

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as preoperative mapping, computer vision, real-time EM tracking, and robot command data may be used individually or in combination to achieve a radiation-free operating environment. In other cases, where radiation-based imaging modalities are still used, the preoperative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart 11 shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Preoperative mapping may be accomplished through the use of the collection of low dose CT scans. Preoperative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images and are particularly appropriate for bronchoscopy.

In some embodiments, the instrument may be equipped with a camera to provide vision data (or image data) 92. The localization module 95 may process the vision data 92 to enable one or more vision-based (or image-based) location tracking modules or features. For example, the preoperative model data 91 may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intraoperatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intraoperatively “registered” to the patient anatomy (e.g., the preoperative model) in order to determine the geometric transformation that aligns a single location in the coordinate system with a position in the preoperative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during preoperative calibration. Intraoperatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Identification of Robotic Arms

Robotic medical systems, such as those described above with reference to FIGS. 1-20 and others, can include a plurality of kinematic chains that include multiple degrees of freedom (such as at least 7 degrees of freedom). These kinematic chains can include a plurality of links and joints. In the illustrated embodiments, the kinematic chains are in the form of robotic arms or robotic manipulators. For example, a robotic medical system can include one or more kinematic chains which can be in the form of robotic arms which can each be configured to control and articulate a medical instrument. For example, the one or more robotic arms of the robotic medical system can include the arms 12 of cart 11 as shown in FIGS. 1-4, the arms 39 of the system 36 as shown in FIGS. 5-6 and 8-10, the arms 50 of system 47 as shown in FIG. 7, the arms 142A of the robotic system 140A of FIG. 14, the robotic arm 76 of FIG. 16, or the robotic arm 82 of FIG. 17. As described herein, the one or more kinematic chains can include any number of kinematic chains such as one, two, three, four, five, six, seven or eight kinematic chains. As noted above, the kinematic chains can be in the form of robotic arms, which accordingly can include any number of arms, such as one, two, three, four, five, six, seven, or eight arms. In the description below, the kinematic chains will be described in in the context of robotic arms which can include a plurality of links and joints.

Each of the robotic arms can be coupled to a medical instrument. For example, a robotic arm can control a medical instrument for a variety of surgical tasks, such as grasping, dissection, cutting, ligation, and/or sealing. During use, each of the robotic arms can each change functions, modes, states, or positions, such as depending on the stage of a procedure. Furthermore, each of the robotic arms can change functions, modes, states or positions based on user preference, the type of procedure, and other factors. Furthermore, the plurality of robotic arms can all be similar in terms of structure. Therefore, users can find it confusing or inconvenient to use features associated with robotic arms that may not be constant as nomenclature. Therefore, it can be advantageous and of clinical significance to use indicators to identify each of the plurality of robotic arms to provide a clear and consistent nomenclature for each of the robotic arms.

Each of the robotic arms can include one or more indicators which can be used to identify each arm. Therefore, the one or more indicators can advantageously identify each arm, regardless of function, mode, state, position, or medical instrument. Such indicators can be particularly useful for efficient communication in the operating room and to reduce use errors. The identification of each arm can provide clear nomenclature for communication between users when referring to different arms. The indicators on each arm can map each arm to associated icons or other representation on the user interfaces. The indicators can advantageously provide clear, distinguishing means for each arm to avoid confusion and minimize cognitive workload for users. As will be explained below, the indicators can also provide means to map to a user interface.

Additionally, users can be positioned in various places relative to the robotic arms. Therefore, the position of each robotic arm can vary depending on the position and perspective of the user. The one or more indicators can allow different users to easily identify each robotic arm in the same manner, regardless of position or perspective. Additionally, the one or more indicators can be positioned or actuated such that any user, regardless of their position in the room, can observe the indicator.

The one or more indicators (such as visual, audible, or haptic indicators, among others) of the robotic arms can be configured to identify each robotic arm as described. Further, the one or more indicators can also provide information regarding each robotic arm to a user or other medical personnel in an operating room. In some examples, the indicators can provide a current state, a function, a mode, a position of the arm, or an associated medical instrument. For example, the indicators can provide the position of the respective robotic arm. The indicators can also provide a type of medical device or a functionality of the medical instrument attached to the respective robotic arm. In some examples, the indicators can provide feedback to the user. For example, the indicators can provide feedback of successful task completion (such as latching, cannula docking, or instrument connection) or of an error or warning (incorrect latching, unsuccessful docking, overheating, or collision). In some examples, the indicators can provide which arm is currently coupled to the controller or user interface.

The use of indicators can advantageously allow reliable and understandable communication between users, such as the surgeon and the staff. The method of identification of the robotic arms can be integrated in components already used in the system and thus can be comfortably used by the users. The use of indicators can also be convenient and be observable by any user in the room. The indicators can advantageously be used in a number of ways and positioned on various components of the robotic system. For example, users can already be trained to look at indicators to provide feedback to the observer regarding the status of the corresponding robotic arm or the system.

The indicators can be positioned in various places. In some examples, the indicators can be positioned on each of the robotic arms. The indicators can be positioned on the distal portions of the robotic arms, which can provide for better visibility. In some examples, the indicators can be permanent attachments to their locations. In some examples, the indicators can be positioned as external components that can be attached to their locations, which can allow for removal for servicing or replacement in case of failure. Furthermore, the indicators can be external components that can be attached to their locations to allow for flexibility of positioning. In some examples, the indicators can be positioned a bar, rail, or arm support that supports the robotic arms.

The illustrated examples of the indicators are provided by way of example, not limitation. For example, the indicators may also be various colors, shapes, symbols, words, or text. In some examples, the indicators can be configured to display text, symbols, images, etc. For example, the indicator can comprise a matrix of individually addressable light sources (e.g., LEDs) or any other type of screen (e.g., an LCD, an LED, or an OLED screen). Such an indicator may be capable of providing more complex information, such as text or diagrams, to a user.

Further, not all indicators need be included in all embodiments. The illustrated embodiments are provided by way of example and illustration and are not intended to be limiting. Upon consideration of this disclosure, one of skill in the art will appreciate that other configurations and embodiments, which are within the scope of this disclosure for systems with indicators are possible. Further, several notable advantages of the indicators for use with robotic medical systems will be described below. Not all of the described advantages need be provided by every embodiment, and the indicators may also provide advantages that are not described herein. Furthermore, while the indicators shown herein are visual indicators, the indicators can also include one or more audible speakers or haptic indicators.

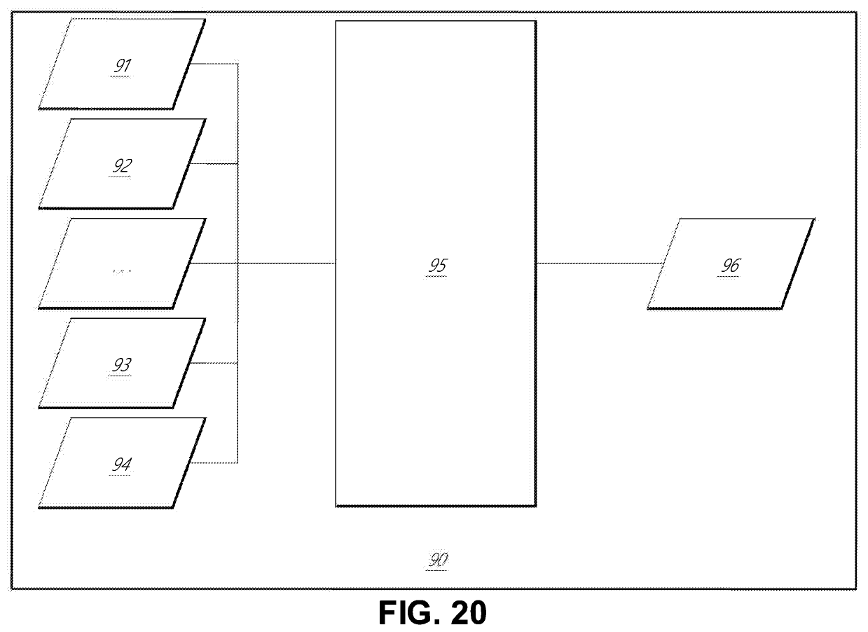

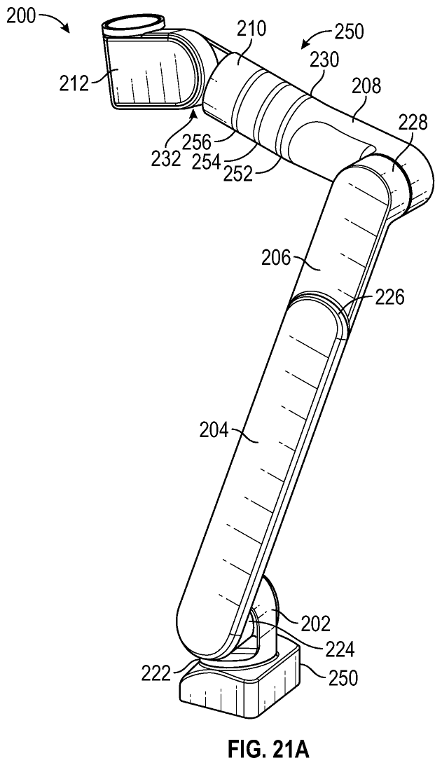

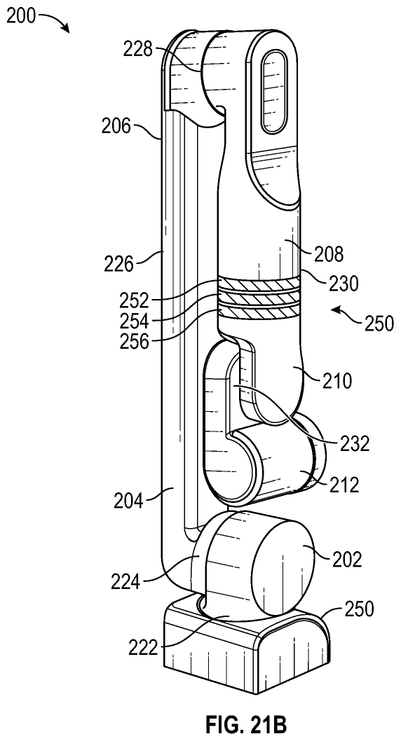

FIGS. 21A-21B illustrates an exemplary embodiment of a kinematic chain with multiple degrees of freedom and a plurality of links and joints. In the illustrated embodiment, the kinematic chain is in the form of a robotic arm 200 that may include an indicator 250. FIG. 21A illustrates the robotic arm 200 in a first extended position. FIG. 21B illustrates the robotic arm in a second folded position. In the illustrated embodiment, the indicator 250 on the robotic arm 200 includes one or more lights (e.g., LEDs or LED arrays). In the illustrated embodiment, the robotic arm 200 can include an indicator 250 in the form of bands or strips that include one or more lights. Although described as including LEDs, other types of light-based indicators can also be used. The indicators 250 can be circumferential rings or discrete lights arranged in a ring pattern that are positioned around the central axis of the robotic arm. In some examples, a light diffuser such as a light guide can be used to distribute the light in a ring fashion around the central axis of the robotic arm. The ring of light can be positioned around a circumference of robotic arm 200, which can allow the for the light to be visible at 360 degrees around the robotic arm 200.

The indicator 250 can include a series of bands, such as a first band 252, a second band 254, and a third band 254. The series of bands 252, 254, 256 can be located in close proximity to each other on the robotic arm. The series of bands 252, 254, 256 can positioned on the distal end of a robotic arm.

The series of bands 252, 254, 26 can be individually addressable such that they can be controlled individually. In some embodiments, different portions or regions, such as individual bands or portions of the bands, of the indicator 250 can be activated (e.g., lit up) or deactivated (e.g., turned off) individually. In some embodiments, the bands 252, 254, 256 of each indicator 250 may be configured to light up in different configurations and different colors. Further, in some embodiments, the bands 252, 254, 256 of the indicator 250 may be configured to light up in different colors and/or to provide illumination at different patterns, colors, brightness, or intensities. Each of the series of bands 252, 254, 256 can illuminate in all visible wavelengths. For example, the bands 252, 254, 256 can light up using discrete RGB LEDs arranged in a ring pattern. In some embodiments, the visual indicators can be configured to change patterns (e.g., a blinking or flashing pattern) and/or change intensity or brightness. Different shading or cross-hatching has been used to illustrate different indicators that can be provided by the indicators (e.g., indications of different colors).

For example, only one of the series of bands 252, 254, 256 can be lit. In some examples, only two of the series of bands 252, 254, 256 can be lit. In some examples, all three of the bands 252, 254, 256 can be lit. In some examples, each of the series of bands 252, 254, 256 can be lit as different colors. Furthermore, each of the three bands 252, 254, 256 can be configured to be lit in different patterns. Therefore, any combination of bands, colors, and/or patterns of the bands 252, 254, 256 can be used to uniquely identify the robotic arm 200.

The robotic arm 200 can be a kinematic chain with multiple degrees of freedom (such as at least 7 degrees of freedom) that can include a base 250 and a plurality of links and joints. For example, in the illustrated embodiment shown in FIGS. 21A-21B, the robotic arm 200 can include six links, a first link 202, a second link 204, a third link 206, a fourth link 208, a fifth link 210, and a sixth link 212. The robotic arm 200 can also include a plurality of joints that connect the base 250 and the links 202, 204, 206, 208, 210, 212. The robotic arm 200 can include a first joint 222, a second joint 224, a third joint 226, a fourth joint 228, a fifth joint 230, and a sixth joint 232. The first joint 222 can be a rolling or roll joint that connects the base 50 with the first link 202. The second joint 224 can be a pitching or pitch joint that connects the first link 202 and the second link 204. The third joint 226 can be a telescoping joint that connects the third link 204 and the fourth link 206 in a telescoping fashion. The fourth joint 228 can be a pitching or pitch joint that connects the third link 206 and the fourth link 208. The fifth joint 230 can be a rolling or roll joint that connects the fourth link 208 and the fifth link 210. The sixth joint 232 can be a pitching or pitch joint that connects the fifth link 210 and the sixth link 212.

The indicator 250 can be included on any of the plurality of links, on any of the links, or the base of the robotic arm 200. The indicator 250 of the robotic arm 200 can be positioned at a distal end of the robotic arm 200. In the illustrated embodiment of FIGS. 21A and 21B, the indicator 250 of the robotic arm 200 includes a series of bands 252, 254, 256 that are positioned about the circumference of the arm 200 positioned along the fifth link 232, between the fifth joint 230 and the sixth joint 232.

The indicator 250 can be positioned on an outer surface of the robotic arm 200. The indicator 250 can be positioned about the circumference of the robotic arm 200. Thus, the indicator 250 can “wrap around” the robotic arm 200. This placement can provide good visibility to users standing anywhere in the room. In some embodiments, the indicator 250 can wrap around multiple surfaces of the robotic arm 200 that can be visible to users positioned at different locations around the robotic arm 200. Also, although the indicator 250 is illustrated as a continuous strip in FIGS. 21A-21B, this need not be the case in all embodiments. Other locations for the indicator 250 are also possible (such as any of the other surfaces) and can be used in place of or in addition to the indicator 250 as illustrated.

FIG. 22 illustrates another exemplary embodiment of a robotic arm 200 that may include an indicator 260. In the illustrated embodiment, the indicator 260 on the robotic arm 200 includes a symbol, which can be a number, letter, word, pattern, or various other shapes. In some examples, each of the robotic arms in the system can be labeled numerically and sequentially. For example, as shown in FIG. 22, the indicator 260 of the robotic arm includes the number “2,” which users can refer to the arm as the second robotic arm. In some embodiments, the indicator 260 can further include a ring surrounding the symbol for further highlighting. In the illustrated embodiment of FIG. 22, the indicator 260 of the robotic arm 200 is positioned on the third link 206 near the fourth joint 228.

In some embodiments, the indicator 260 can be static. In some embodiments, the indicator 260 can also include one or more lights such that at least a portion of the indicator 260 can be illuminated. The indicator 260 can include LEDs or other types of lights. The individual components of the indicator 260, such as the ring and the symbol, can be individually addressable such that they can be controlled individually. Thus, in some embodiments, different portions or regions of the indicator 260 can be activated (e.g., lit up) or deactivated (e.g., turned off) individually. Further, in some embodiments, the ring and symbol of the indicator 260 may be configured to light up in different colors and configurations and/or to provide illumination at different patterns or intensities.

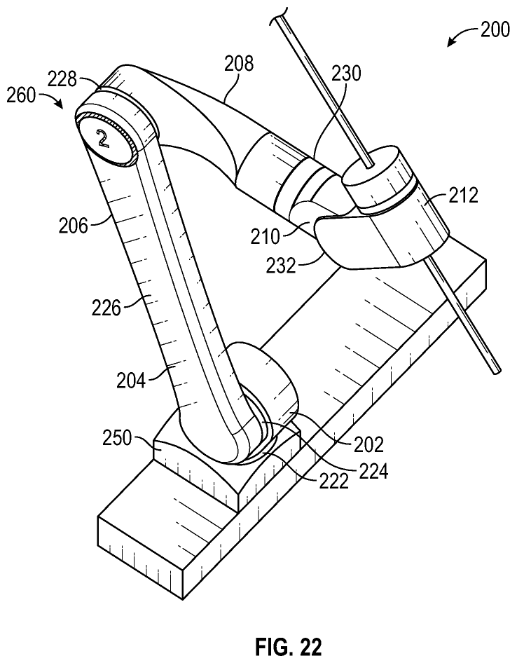

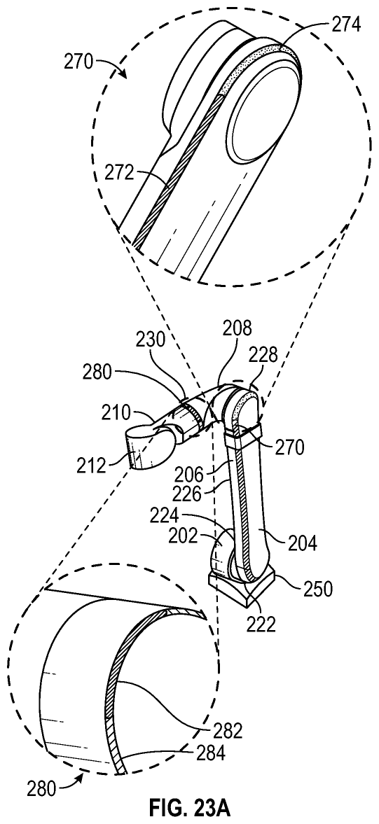

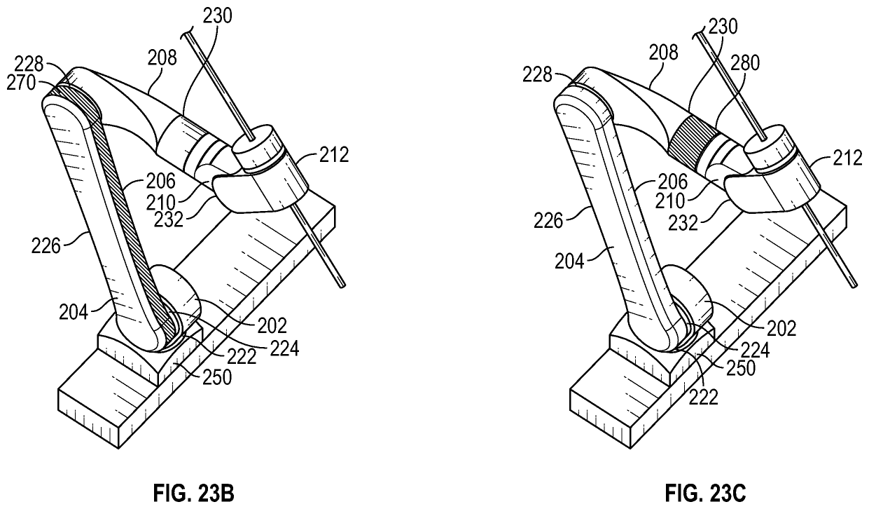

FIG. 23A illustrates another exemplary embodiment of a robotic arm 200 that may include a first indicator 270 and a second indicator 280. FIG. 23B illustrates an exemplary robotic arm 200 that includes the first indicator 270. FIG. 23C illustrates an exemplary robotic arm 200 that includes the second indicator 270. In the illustrated embodiment, the first indicator 270 and/or the second indicator 280 on the robotic arm 200 includes one or more lights (e.g., LEDs or LED arrays). In the illustrated embodiment, the robotic arm 200 can include the first indicator 270 and/or the second indicator 280 in the form of bands or strips that include one or more lights. The first indicator 270 and/or the second indicator 280 can light a particular color or pattern to identify the respective robotic arm, similar to the indicator 250 described in FIGS. 21A-21B.

In the embodiment FIG. 23A, with both the first indicator 270 and the second indicator 280 present on the robotic arm, the first indicator 270 and/or the second indicator 280 can be individually addressable such that they can be controlled individually. Furthermore, the first indicator 270 can have a first or proximal portion 272 and a second or distal portion 274 that can be controlled individually. Similarly, the second indicator 280 can have a first portion 272 and a second portion 284 that can be controlled individually. The first indicator 270 and/or the second indicator 280 can be configured to light up in different configurations and different colors, which can provide variability to further differentiate the robotic arms as well as providing different types of information. In some examples, the first indicator 270 and the second indicator 280 can be lit the same color or pattern to identify the respective robotic arm. The use of multiple indicators can provide reinforcement of color identity at various locations on the arm and further enhance visibility of the indicators from various positions in the room.

In the illustrated embodiment of FIGS. 23A and 23B, the first indicator 270 of the robotic arm 200 is positioned along the length of the second link 204 and/or the third link 206. The first indicator 270 can be positioned at the edge or side of the second link 204 and/or the third link 206. In the illustrated embodiments of FIGS. 23A and 23C, the second indicator 280 of the robotic arm 200 can be positioned around a circumference of the fifth link 210, similar to the indicators 250 as shown in FIGS. 21A-21B.

FIGS. 21A-21B, 22, and 23A-23C are intended to provide examples of configurations and placement locations for indicators on the robotic arm 200. The illustrated embodiments are not intended to be limiting and other locations and placements for the indicator 250 on the robotic arm 200 are also possible. In the alternative, or in addition, other types of combinations indicators may be placed on one or more portions of the robotic arm 200. For example, the indicators can be placed on any one of the plurality of links, any one of the joints, and/or the base.

As mentioned above, the indicators described above can be configured to communicate information about the robotic medical system to users. Such information can comprise state or identity information for the system. As used herein, “state information” refers broadly to any information indicative of a state or status or condition of the robotic medical system or a component thereof “Identity information” is also used broadly to refer to information that can be used to identify or differentiate a component of a robotic medical system, such as a particular robotic arm.

The indicators can be programmed for user preference. This can allow a user to use their preferred method of using the indicators to differentiate the robotic arms. For example, the user can program the indicators to illuminate particular colors, patterns, or intensities in a desired order.

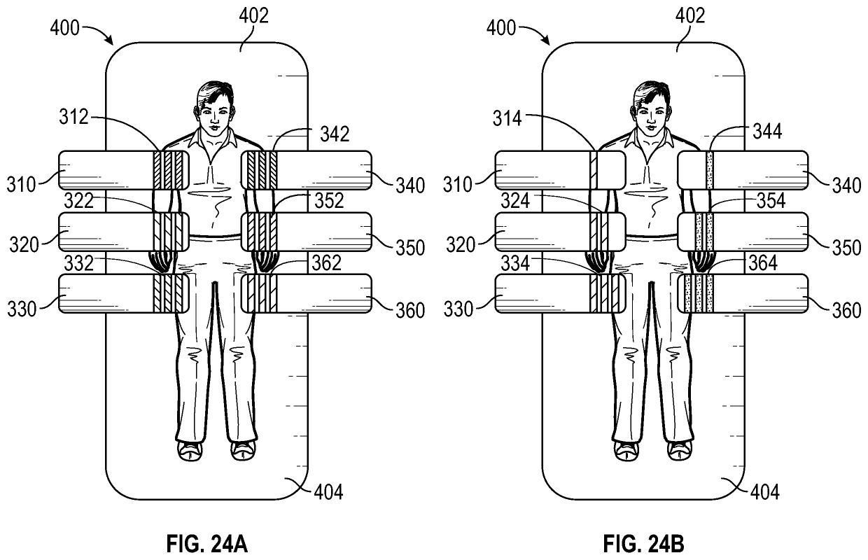

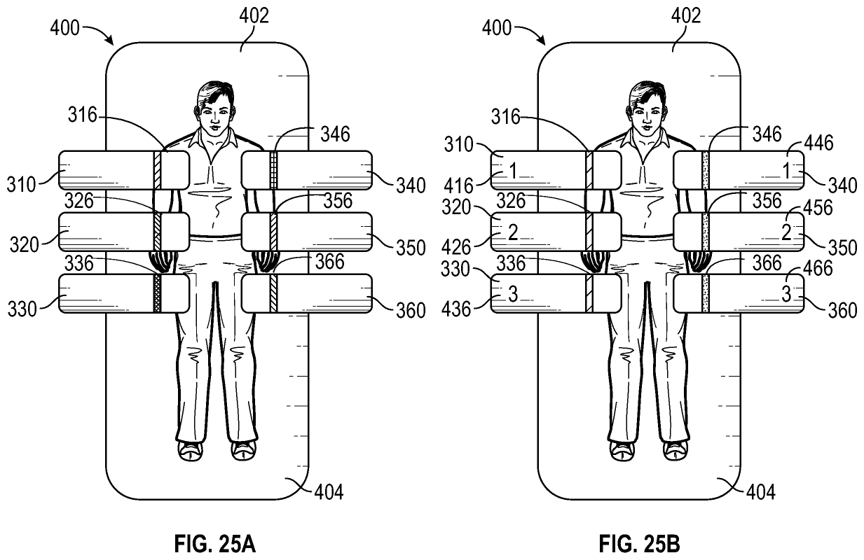

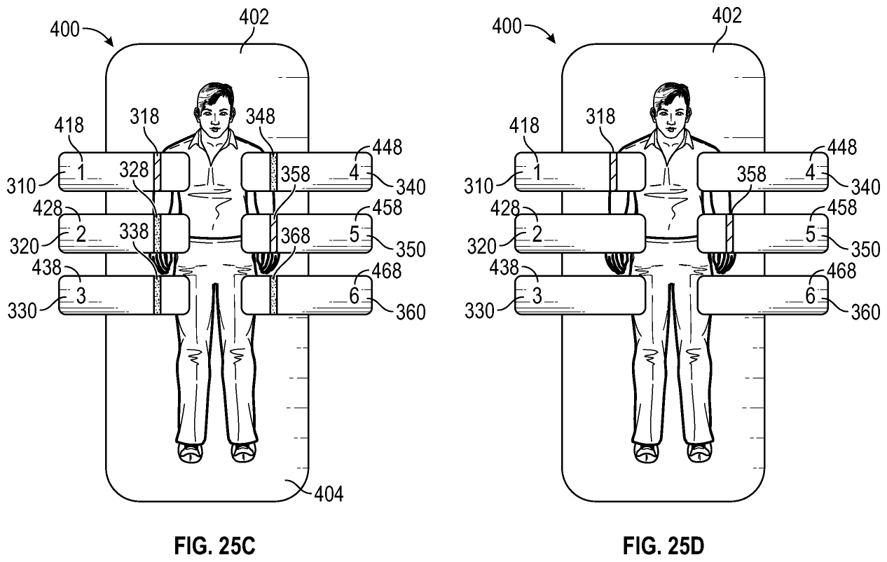

FIGS. 24A-24B and 25A-25D are top views of the robotic medical systems 400 illustrating functionality that can be provided by indicators. In these figures, only the patient platform 400 and the robotic arms 310, 320, 330, 340, 350, 360 are illustrated. Additional features have been omitted for clarity. The first, second, and third robotic arms 310, 320, 330 can be positioned on a first side of the patient platform 400. The fourth, fifth, and sixth robotic arms 340, 350, 360 can be positioned on a second side of the patient platform 400, the second side being opposite the first side.

As shown in FIG. 24A, in some embodiments, each of the robotic arms 310, 320, 330, 340, 350,360 can include a set of indicators 312, 322, 332, 342, 352, 362, respectively. Each of the set of indicators 312, 322, 332, 342, 352, 362, can each include three bands, similar to the indicators 250 the robotic arm 200 shown in FIGS. 21A-21B. Each of the set of indicators 312, 322, 332, 342, 352, 362 can be configured to light up as different colors, intensities, or patterns, to distinguish from one another. As described above, each of the set of indicators 312, 322, 332, 342, 352, 362 can each be configured to light up a different number of bands.

The total number of bands on each robotic arm can be based on the total number of robotic arms in the system. For example, the number of bands on each robotic arm can equal half the total number of arms in the system. As shown in FIG. 24A, there are three bands on each robotic arms and a total of six robotic arms in the system. In other embodiments in which eight robotic arms are used in the system, each robotic arm would include four bands.

As shown in FIG. 24B, in some embodiments, each of the robotic arms 310, 320, 330, 340, 350,360 can include a set of indicators 314, 324, 334, 344, 354, 364, respectively. The first, second, and third arms 310, 320, 330 can be positioned on the first side of the patient platform 400. The first indicator 314 of the first arm 310 can include a single band. The second indicator 324 of the second arm 320 can include two bands. The third indicator 334 of the third arm 330 can include three bands. The fourth, fifth, and sixth arms 340, 350, 360 can be positioned on the second side of the patient platform 400. The fourth indicator 344 of the fourth arm 340 can include a single band. The fifth indicator 354 of the fifth arm 350 can include two bands. The sixth indicator 364 of the sixth arm 360 can include three bands.

Each of the indicators 314, 324, 334 of the first, second and third arms 310, 320, 330 on the first side of the platform 400 can be configured to light the same color, pattern or intensity. Each of the indicators 344, 354, 364 of the fourth, fifth, and sixth arms 340, 350, 360 on the second side of the platform 400 can be configured to light the same color, pattern or intensity.

The indicators of each arm on one side of the platform 400 have a different number of bands (e.g. the first arm 310, the second arm 320, and the third arm 330 are all positioned on a first side of the platform 400 and each of their respective indicators have a different number of bands). The number of bands of each indicator can indicate the position of the robotic arm, such as in a distal-proximal direction of the platform 400. The platform 400 can have a proximal end 402 and a distal end 404. The robotic arms positioned on the proximal end 402 of the platform 400, the first arm 310 and the fourth arm 340, can have a single band indicator. The robotic arms positioned in the middle of the platform 400, the second arm 320 and the fifth arm 350, can have two band indicators. The robotic arms positioned on the distal end 404 of the platform 400, the third arm 330 and the sixth arm 360, can have three band indicators.

In this configuration, the robotic arms on each side of the platform 400 can be differentiated based on color. In this configuration, each of the robotic arms on single side of the platform 400 can be differentiated by a different number of rings on each robotic arm.

Additionally, the system of FIG. 24A can also achieve this same identification pattern as described of FIG. 23B. For example, the first indicators 312 of the first arm 310 and the fourth indicators 342 of the fourth arm 340 can be configured to light a single band of the series of bands. For example, the second indicators 322 of the second arm 320 and the fifth indicators 352 of the fifth arm 350 can be configured to light two bands of the series of bands. For example, the third indicators 332 of the third arm 330 and the sixth indicators 362 of the sixth arm 360 can be configured to light three bands of the series of bands.

In some instances, colors and patterns may not be easily distinguishable from one another in the same way to all users. For example, one individual might see a blue band while another user sees a green band. Furthermore, it may take time and effort to observe different patterns. For example, it may take a few seconds for a user to observe a flashing or blinking pattern of a particular indicator. The confusion can further be exacerbated by the drapes that can be positioned over the robotic arms used to maintain sterility. The use of drapes can make it difficult to observe patterns, intensity, and colors. For example, colors emitted by the lights can be seen in a different wavelength once they reach the viewers eye through the drape. Therefore, the use of the number of band of indicators in addition to colors and patterns can advantageously allow a user to quickly and easily understand the indicators.

The use of multiple distinguishing feature such as colors, patterns, and number of bands, either alone or in combination, allows for flexibility to identify each robotic arm. For example, some users may wish to identify each robotic arm with a different color, while other users may prefer to use the number of bands to identify each robotic arm. Furthermore, the use of multiple distinguishing features can also allow for redundancy to ensure all users are able to clearly and quickly understand identification of each robotic arm.

Furthermore, the use of multiple distinguishing features, allows for multiple pieces of information to be conveyed. For example, in one embodiment, the different number of bands can be used to identify the arms and the different colors can be used for the type of medical instrument attached to each robotic arm. For example, the intensity can be used to show the state of the robotic arm, such as whether the robotic arm is active or not active.

As shown in FIG. 25A, each of the robotic arms 310, 320, 330, 340, 350, 360 can include a set of indicators 316, 326, 336, 346, 356, 366, respectively. Each of the set of indicators 316, 326, 336, 346, 356, 366, can each include one band. Each of the set of indicators 316, 326, 336, 346, 356, 366 can be configured to light up as different colors, intensities, or patterns. The use of different indicators can distinguish the robotic arms from one another and/or provide other information.

As shown in FIG. 25B, in addition to the first type of indicators 316, 326, 336, 346, 356, 366, respectively, each of the robotic arms 310, 320, 330, 340, 350, 360 can also include a second type of indicators 416, 426, 436, 446, 456, 466, respectively. The second type of indicators 416, 426, 436, 446, 456, 466, can include a number or symbol to distinguish each of the robotic arms 310, 320, 330, 340, 350, 360 from one another. The second type of indicators can be positioned in one or more locations of the robotic arms, such as on a proximal link, distal link, joints, or combinations thereof. Each of the second type of indicators 416, 426, 436, 446, 456, 466, can include a different number or symbol to differentiate or identify each of the robotic arms, similar to the indicator 260 as shown in FIG. 22. The first type of indicators can be considered distal indicators, as they are positioned near a distal end of the robotic arms. The second type of indicators can be considered proximal indicators as they are positioned near the proximal end of the robotic arms. In some embodiments, the second type of indicators are static or dynamic.

The first type of indicators 316, 326, 336, 346, 356, 366, and the second type of indicators 416, 426, 436, 446, 456, 466, can each be used to distinguish each of the robotic arms 310, 320, 330, 340, 350, 360 from one another. Using both types of indicators can provide redundancy in the system which can allow colorblind users to identify the robotic arms. When both types of indicators are used, the numbers can be repeated on both sides of the platform 400. For example, as shown in FIG. 24B, the second indicator 416 first arm 310 and the second indicator 446 of the fourth arm 340 can each include the number “1,” the second indicator 426 of the second arm 320 and the second indicator 456 of the fifth arm 350 can each include the number “2,” and the second indicator 436 of the third arm 330 and the second indicator 466 of the sixth arm 360 can each include the number “3.” In another embodiment, the robotic arms can use both types of indicators (e.g., a first type of indicator comprising one or more bands of light and a second type of indicator comprising a number identification scheme) in parallel, whereby half of the robotic arms use bands of light of a first color and the other half of the robotic arms use bands of a light of a second color. In this scenario, the number of colors used m in less than the number of different numeric identifiers n (n=3m). Advantageously, by having two indicators of different numbers, this helps ease the level of complexity but still manages to clearly communicate information between users.

In some examples, the first type of indicators 316, 326, 336, 346, 356, 366 can be used to convey a status or position of the respective robotic arm. For example, as illustrated in FIG. 24B, each of the first indicators 316, 326, 336 of the first, second and third arms 310, 320, 330 on the first side of the platform 400 can be configured to light the same color, pattern or intensity. Each of the first indicators 346, 356, 366 of the fourth, fifth, and sixth arms 340, 350, 360 on the second side of the platform 400 can be configured to light the same color, pattern or intensity. In this configuration, the robotic arms on each side of the platform 400 can be differentiated based on color of the respective first indicators. The color of the respective first indicators 316, 326, 336, 346, 356, 366 can be used to convey the position (such as a first side or a second side, the proximal end 402 and the distal end 404 of the patient platform 400) of the respective robotic arm.

In some examples, the user could identify each arm by its color of the first type of indicators 316, 326, 336, 346, 356, 366 and its color of the second type of indicators 416, 426, 436, 446, 456, 466. For example, the first indicators 316, 326, 336 of the first, second and third arms 310, 320, 330 on the first side of the platform 400 can be configured to light a first color, such as blue. Each of the first indicators 346, 356, 366 of the fourth, fifth, and sixth arms 340, 350, 360 on the second side of the platform 400 can be configured to light a second color, such as green. Thus, a user could call the first robotic arm 310 as “blue 1,” the second robotic arm 320 as “blue 2” and the third robotic arm 330 as “blue 3,” the fourth robotic arm 340 as “green 1,” the fifth robotic arm as “green 2,” and the sixth robotic arm as “green 3.” The use of two types of indicators could advantageously provide redundancy in the system, allow for colorblind users to identify the robotic arms, reduces cognitive load, and minimizes risk during use.

As shown in FIG. 25C, the second type of indicators 418, 428, 438, 448, 458, 468, can include a different number or symbol to distinguish each of the robotic arms 310, 320, 330, 340, 350, 360 from one another. For example, as shown in FIG. 25C, the second indicator 418 on the first arm 310 can include the number “1,” the second indicator 425 on the second arm 320 can include the number “2,” the second indicator 438 on the third arm 330 can include the number “3,” the second indicator 448 on the fourth arm 340 can include the number “4,” the second indicator 458 on the fifth arm 350 can include the number “5,” and the second indicator 418 on the sixth arm 360 can include the number “6.” The first type of indicators 318, 328, 338, 348, 358, 368 can be used to convey an identity. The first type of indicators 318, 328, 338, 348, 358, 368 can also be used to indicate a state or function of the respective robotic arm. For example, as illustrated in FIG. 24C, the indicators 318 and 358 can be configured to light a first color, pattern or intensity, while the indicators 328, 338, 348, 368 can be configured to light a second color, pattern or intensity. In this configuration, the first color can indicate the associated robotic arms are in a first state, such as active, while the second color can indicate the associated robotic arms are in a second state, such as inactive. In this configuration, the first color can indicate the associated robotic arm are working and fault-free, while the second color can indicate the associated robotic arms have an error. Other types of information may be conveyed through the indicators, such as the mode or functionality of the associated robotic arm. In some examples, the indicators can indicate whether a particular robotic arm is coupled to a user input, the type of medical instrument coupled to the robotic arm, or whether there is an error or warning associated with the robotic arm.

As shown in FIG. 25D, only the respective first type of indicators of active robotic arms could be lit to indicate a state or mode, such as an active state. For example, as shown in FIG. 24D, only the first indicator 318 of the first robotic arm 310 and the first indicator 358 of the fifth robotic arm 350 can be lit, which could indicate only the first robotic arm 310 and the fifth robotic arm 350 are in that state. The lack of lights or colors in the other indicators of the remaining robotic arms would indicate they are in a second state, different from the first state, such as inactive. This advantageously reduces the visual noise and simplifies the indicators further to a user.

The use of multiple distinguishing features, colors, patterns, number of bands, allows for flexibility to identify each robotic arm or to communicate information regarding each robotic arm. For example, some users may wish to identify each robotic arm with a different color, while other users may prefer to use the number of bands to identify each robotic arm. This also allows for flexibility for users to program the indicators on the robotic arms for their preferred uses. Furthermore, the use of multiple distinguishing features can also allow for redundancy to ensure all users are able to clearly and quickly understand identification of each robotic arm.

Furthermore, the use of multiple distinguishing features, allows for multiple pieces of information to be conveyed. For example, the different number of bands can be used to identify the arms and the different colors can be used for the type of medical instrument attached to each robotic arm. For example, the intensity can be used to show the state of the robotic arm, such as whether the robotic arm is active or not active.

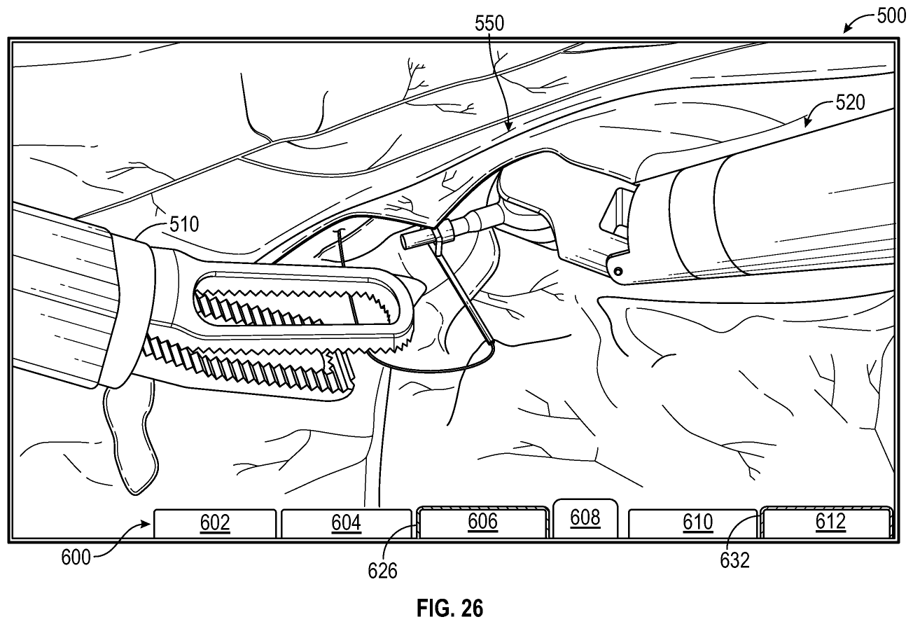

The indicators of the robotic arms as described above can be mapped a user interface. FIG. 26 illustrates a display 500 that can include a rendering of an image or representation (graphical or otherwise) of one or more medical instruments 510, 520 in a surgical site 550. The display 500 can also include a series of tabs or a menu 600 as image overlays positioned over the image of the one or more medical instruments 510, 520 on the surgical site 550. The series of tabs 600 can include a first tab 602, a second tab 604, a third tab 606, the fourth tab 608, and a fifth tab 610, and a sixth tab 612. Each of the tabs can correspond to a different robotic arm. Each of the tabs can have a feature (such as a color or pattern) corresponding to the indicator of the respective robotic arm.

In some examples, each of the series of tabs 600 can include an image overlay indicator positioned around at least a portion of the tab. For example, as shown in FIG. 26, the third tab 606 includes a third image overlay indicator 626 positioned around a portion of the perimeter of the third tab 606 and the sixth tab 612 includes a sixth image overlay indicator 632 positioned around a portion of the perimeter of the sixth tab 612. The image overlay indicators 626, 632 can have a color or pattern corresponding to the color or pattern of the indicator positioned on the respective robotic arm. For example, if the third robotic arm includes an indicator (such as indicator 250 of FIGS. 21A-21B) that is a first color, the third tab 626 can be the same first color and/or include a highlighted portion 626 of the first color. This will allow a user to easily identify which tab is associated with which robotic arm.

### 3. Implementing Systems and Terminology

Implementations disclosed herein provide systems, methods and apparatus associated with indicators configured for use with robotic medical systems.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

Any phrases referencing specific computer-implemented processes/functions described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A medical robotic system comprising:
a first kinematic chain comprising a first set of indicators; and
a second kinematic chain comprising a second set of indicators,
wherein actuation of the first set of indicators or the second set of indicators differentiates the first kinematic chain from the second kinematic chain.

2. The medical robotic system of claim 1, wherein the first kinematic chain and the second kinematic chain comprise an equal number of degrees of freedom.

3. The medical robotic system of claim 2, wherein the equal number of degrees of freedom comprises at least 7 degrees of freedom.

4. The medical robotic system of claim 1, wherein the first kinematic chain is a first robotic arm and the second kinematic chain is a second robotic arm.

5. The medical robotic system of claim 4, wherein the first robotic arm and the second robotic arm are supported on an arm support.

6. The medical robotic system of claim 1, wherein the first set of indicators are positioned at a distal end of the first kinematic chain and the second set of indicators are positioned at a distal end of the second kinematic chain.

7. The medical robotic system of claim 1, wherein the first set of indicators and the second set of indicators each comprise two or more bands of light.

8. The medical robotic system of claim 7, wherein the two or more bands of lights of the first set of indicators are each positioned about a circumference of the first kinematic chain, and wherein the two or more bands of lights of the second set of indicators are each positioned about a circumference of the second kinematic chain.

9. The medical robotic system of claim 8, wherein each band of light is visible at 360 degrees about its respective first kinematic chain and/or the second kinematic chain.

10. The medical robotic system of claim 7, wherein, on the first kinematic chain, the two or more bands of lights are positioned between a roll joint and a pitch joint.

11. The medical robotic system of claim 1, wherein the first kinematic chain comprises six links and six joints.

12. The medical robotic system of claim 11, wherein the first set of indicators are positioned on a fifth link of the six links.

13. The medical robotic system of claim 12, wherein the first set of indicators are positioned between a fifth joint and a sixth joint of the six joints.

14. A medical robotic system comprising:
a plurality of robotic arms, wherein each of the plurality of robotic arms comprises one or more ring indicators,
wherein actuation of the plurality of indicators differentiates each of the plurality of robotic arms.

15. The medical robotic system of claim 14, wherein the one or more indicators comprise a number of ring indicators equal to half a number of the plurality of arms.

16. The medical robotic system of claim 14, wherein the one or more ring indicators are configured to display multiple colors, and wherein the one or more ring indicators of each of the plurality of robotic arms are configured to display a different color.

17. The medical robotic system of claim 14, wherein the plurality of robotic arms comprises a first set of robotic arms positioned on a first side of a bed, and wherein the plurality of robotic arms comprises a second set of robotic arms positioned on a second side of the bed.

18. The medical robotic system of claim 17, wherein the one or more ring indicators of each of the first set of robotic arms are configured to display a first color, wherein the one or more ring indicators of each of the second set of robotic arms are configured to display a second color, the second color different from the first color.

19. The medical robotic system of claim 17, wherein each of the first set of robotic arms are configured to display a different number of ring indicators to differentiate each of the first set of robotic arms.

20. The medical robotic system of claim 14, wherein the one or more ring indicators of each of the plurality of robotic arms are configured to be programmed by a user.
